(19) United States
(12) Patent Application Publication
(10) Pub. No.: US 2025/0166818 A1
(43) Pub. Date: May 22, 2025

(54) Clinical Decision Tool

(71) Applicant: Brendan Ruby, Boston, MA (US)

(72) Inventor: Brendan Ruby, Boston, MA (US)

(21) Appl. No.: 18/515,197

(22) Filed: Nov. 20, 2023

(51) Int. Cl. G16H 50/20

(52) U.S. Cl. CPC G16H 50/20

(57) ABSTRACT

Clinical Decision Tool or computer process centered on Consequentialism to solve difficult hospital decisions. Decisional dilemmas in hospitals can be solved through use of clinical decision tools. Consequentialism is central to moral decisions. Connecting Consequentialism moral concept to a clinical decision tool solves moral dilemmas in hospitals.

### TECHNICAL FIELD

The present disclosure relates to the field of medical engineering and artificial intelligence.

### BACKGROUND

Outcomes in hospital settings are often caused by doctors' decisions. To improve outcomes in hospitals, one must improve doctors decisional processes. Concepts in bioethics literature such as Consequentialism can assist on producing moral decisions. Consisting of prioritizing the outcome of a decision over the initial harm that a decision may produce, is Consequentialism. A clinical example is providing a treatment to a patient that may be initially harmful but produces an overall good consequence such as a cured disease.

### SUMMARY

The presently disclosed technology teaches a medical system for automated determination of an optimized treatment protocol and dynamic allocation of medical resources, based on the principle of consequentialism.

### DETAILED DESCRIPTION

In order to more clearly illustrate the technical solutions of the embodiments of the present disclosure, the accompanying drawings for the description of the embodiments are described below. Obviously, the accompanying drawings in the following description are only some examples or embodiments of the present disclosure, and it is possible for a person of ordinary skill in the art to apply the present disclosure to other similar scenarios in accordance with these accompanying drawings without creative labor. Unless obviously obtained from the context or the context illustrates otherwise, the same numeral in the drawings refers to the same structure or operation.

It should be understood that the terms “system,” “device,” “unit,” and/or “module” are used herein as a way to distinguish between different components, elements, parts, sections, or assemblies at different levels. However, if other words may achieve the same purpose, the terms may be replaced with alternative expressions.

As indicated in the present disclosure and in the claims, unless the context clearly suggests an exception, the words “one,” “a,” “a kind of,” and/or “the” do not refer specifically to the singular but may also include the plural. In general, the terms “include” and “comprise” suggest only the inclusion of clearly identified steps and elements, which do not constitute an exclusive list, and the method or device may also include other steps or elements.

Centered on the concept of Consequentialism and connected to a clinical decision tool used by doctors, our computer process provides sound solutions to those who provide necessary information to our process. Connecting the central concept of Consequentialism to a Clinical Decision Tool would solve decisional dilemmas in hospitals. To outline this step by step, it starts with a doctor that comes across a complex decisional dilemma. Second the doctor then provides necessary clinical information to our computer process such as circumstances of the medical decision, clinical information of patient, and options of care. Next the online computer software or process processes the information through a filter of moral principle of Consequentialism, to show the doctor a solution to the moral dilemma initially posed. Our computer process filters the initial input of the doctor or options of care presented through the concept of Consequentialism, a concept known in bioethics literature. Consequentialism holds how one should act in a specific way with a priority on the outcome instead of the initial action itself. It is a moral concept known across the academic world. Some scenarios in hospitals of consequentialism is Treatment Options, End of Life Care, Resource Allocation, Organ Transplant, or Research or Clinical Trials. Consequentialism should guide decision making in hospitals, yet no clinical decision tools integrate this central concept into providing decisions to doctors. Integrating the central concept of consequentialism could assist in treatment options; showing doctors how the outcomes or consequences of each option influence the overall quality of life of the patient, with a specific lens on consequentialism as a principle of morality. Complex clinical decisions require use of this principle, yet no clinical decision tool currently offers its use in providing a comprehensive solution to doctors. Two patients in need of a specific medical device poses a complex moral decision to a doctor who could ask our computer process to solve. Two patients in need of the device, the device should be assigned to the patient whose condition is more critical and whose outcomes hold more positives than the other patients outcomes or consequences of the treatment. The computer process processes information such as the patients clinical situation, such as age, weight, diagnoses, genetics, and device availability in that specific hospital. A doctor asks or inputs the relevant decisional dilemma and receives a consequentialist solution through this computer process. Artificial Intelligence could assist on this process and could be a central component. Filters doctors input through concept of Consequentialism to solve complex decisional dilemmas in hospitals.

Outcomes of this tool involve producing sound decisions to doctors coinciding by the specific principles of consequentialism, a concept widely known in bioethics. The whole process occurs on a computer. Specific clinical data of patient is provided by doctor or hospital worker, data is processed through artificial intelligence and through the concept of consequentialism, solution or decision is the outcome of this computer process, displaying on electronic screen a solution or decision to initial dilemma—coinciding by principles of consequentialism.

Consists of Artificial Intelligence to process necessary clinical data using the principles of Consequentialism, a concept in bioethics, as a decision tool in hospitals. Posed with a dilemma by a doctor or hospital worker, this clinical decision tool processes necessary clinical data with the clinical dilemma to produce a decision coinciding by Consequentialism principles. Process is specific to consequentialism concept. Artificial Intelligence or AI is central to this Clinical Decision Tool, as it processes necessary information in a quick speed to show doctors solutions to their dilemmas in hospitals or clinical situations where decisions must be made quickly and comprehensively and according to specific moral principles. Consequentialism is a moral principle known in bioethics. Through this process, a moral principle such as consequentialism is implemented in hospitals to improve patient outcomes. Its central to be moral to patients.

Outcomes of patients can be augmented through connecting a moral principle (consequentialism) to decisional dilemmas, such as in an Emergency Room in a Hospital, or other departments as necessary.

Clinical Decision Tool or process to assist doctors on decisions in hospitals. On a hospital computer a doctor consults our process in order to solve a decisional dilemma coinciding by a moral principle—consequentialism, to improve the outcome of a decision to be more sound.

Shows doctors options of care or decisions to choose, on case by case specificity in hospitals. Options of care are shown on a computer screen proceeding processing of necessary principles or data.

Connecting Alto a computer, coinciding by principles of consequentialism, in order to solve doctors decisions through a clinical decision tool. This tool can be implemented in any discipline of medicine, such as cardiology, internal medicine, surgery, emergency rooms, pathology, or more. Comprehensively solves decisions to doctors, showing options of care and necessary facts to back up a decision. On a computer, doctor inputs necessary clinical data or circumstances surrounding a decision in a hospital. Solution is quickly or soundly provided through our clinical decision tool or process. Provides reasoning behand the decision or solution, coinciding by consequentialism, to show a moral decision or clinical path. Shows facts or clinical studies or sentences or paragraphs to support the decision or solution.

Decisions often coincide with certain principles or concepts. Yet no clinical decision tool exists that connects AI and Consequentialism to provide a moral solution to a doctor. Doctors can use our process or tool to assist on decisions. Decisions that do not coincide by moral principles can be harmful to patients or hospitals. A filter is a process that lets in certain components and keeps out other components. An example of a filter is a water filter one that filter's out harmful dirt or chemicals and filters in pure drinking water. Using the concept of a filter centered on Consequentialism—keeps out immoral decisions and filters through moral decisions that coincide by Consequentialist principles on case by case specificity in a hospital setting.

Consequentialism is the doctrine that the morality of an action is to be judged solely by its consequences. It is a principle known or used in bioethics or by bioethics committees in hospitals to assist on decisions. Yet it is not electronic or on a computer as a clinical decision tool to produce moral patient outcomes.

### DESCRIPTION

A computer decision tool in hospitals that practices consequentialism. Doctor provides patients' clinical data or options of care or decisional dilemma to this decision tool, next the data goes through a computer filter of consequentialism, and produces a decisional solution to doctor on computer. On a case by case specificity in hospitals, this decision tool can solve complex moral dilemmas or decisional dilemmas in hospitals through the concept of Consequentialism. Holds how an outcome or consequence of an action holds more moral weight than the action itself, even if the initial action produces initial harm. Solves moral dilemmas in hospitals to doctors on a computer.

Furthermore, unless explicitly stated in the claims, the use of order, numbers, letters, or other names for processing elements and sequences are not intended to limit the order of the processes and methods of the present disclosure. While various examples have been discussed in the disclosure as currently considered useful embodiments of the invention, it should be understood that such details are provided for illustrative purposes only. The appended claims are not limited to the disclosed embodiments, and instead, the claims are intended to cover all modifications and equivalent combinations within the scope and essence of the embodiments disclosed in the present disclosure. For example, although the described system components may be implemented through a hardware device, they may also be realized solely through a software solution, such as installing the described system on an existing processing or mobile device.

Similarly, it should be noted that, for the sake of simplifying the presentation of embodiments disclosed in the present disclosure and aiding in understanding one or more embodiments of the present disclosure, various features have been sometimes combined into a single embodiment, drawing, or description. However, this manner of disclosure does not imply that the features required by the claims are more than the features mentioned in the claims. In fact, the features of the embodiments are less than all the features of the single embodiment disclosed in the foregoing disclosure.

In some embodiments, numeric values describing the composition and quantity of attributes are used in the description. It should be understood that such numeric values used for describing embodiments may be modified with qualifying terms such as “about,” “approximately” or “generally”. Unless otherwise stated, “about,” “approximately” or “generally” indicates that a variation of ±20% is permitted in the described numbers. Accordingly, in some embodiments, the numerical parameters used in the disclosure and claims are approximations, which can change depending on the desired characteristics of the individual embodiment. In some embodiments, the numerical parameters should take into account a specified number of valid digits and employ a general manner of bit retention. Although the numerical ranges and parameters used in some embodiments of the present disclosure to confirm the breadth of the range are approximations, in specific embodiments, such numerical values are set as precisely as practicable.

With respect to each of the patents, patent applications, publications of patent applications, and other material, such as articles, books, specifications, publications, documents and the like, cited in the present disclosure, the entire contents thereof are hereby incorporated herein by reference. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terminology in the materials appended to the present disclosure and the contents described herein, the descriptions, definitions, and/or use of terminology in the present disclosure shall prevail.

In closing, it should be understood that the embodiments described in the present disclosure are used only to illustrate the principles of the embodiments of the present disclosure. Other deformations may also fall within the scope of the present disclosure. Therefore, by way of example and not limitation, alternative configurations of the embodiments disclosed in the present disclosure may be considered consistent with the teachings of the present disclosure. Accordingly, the embodiments described in the present disclosure are not limited to the explicitly introduced and described embodiments in the present disclosure.

1-3. (canceled)

4. A medical system for automated determination of an optimized treatment protocol and dynamic allocation of medical resources, comprising:
a clinical database including clinical information of one or more patients, including demographic information, diagnosis, and assessments of each of the one or more patients; wherein the demographic information at least includes age, weight, and genetics;
a plurality of medical devices and medical resources;
a computerized device, including one or more hardware computer processors configured with computer-executable instructions that, when executed, cause the one or more hardware computer processors to:
retrieve a first group of feature data from the clinical database;
gather a second group of feature data regarding a statistic of the plurality of medical devices and medical resources;
gather a third group of feature data regarding options of treatment and care for each of the one or more patients;
display an outcome of the options of treatment and care upon each of the one or more patients to healthcare professionals;
feed the first, second, and third groups of feature data to an artificial intelligence (AI) model;
wherein the AI model operates based on consequentialism, optimizing an aggregation of the outcome of treatment and care for each of the one or more patients;
cause the AI model to evaluate and output a first optimal decision regarding a selected option of treatment and care, from the options of treatment and care, for each of the one or more patients, based on the first, second, and third groups of feature data;
cause the AI model to evaluate and output a second optimal decision regarding an allocation of the resources in the hospital.

5. The medical system in claim 4, wherein the computerized device is further programmed to automatically identify relevant clinical studies, import additional training data from the relevant clinical studies, and automatically revise the AI model based on the additional training data.
